(19) United States
(12) Patent Application Publication
(10) Pub. No.: US 2010/0185398 A1
(43) Pub. Date: Jul. 22, 2010

(54) System and Method for Monitoring Athletic Performance

(75) Inventors: Jason A. Berns, Baltimore, MD (US); William K. Mickle, Baltimore, MD (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: UNDER ARMOUR, INC., Baltimore, MD (US)

(21) Appl. No.: 12/692,528

(22) Filed: Jan. 22, 2010

Related U.S. Application Data
(60) Provisional application No. 61/146,559, filed on Jan. 22, 2009.

(51) Int. Cl. G06F 19/00; A61B 5/00; G06N 5/02; H04L 9/32

(52) U.S. Cl. 702/19; 600/301; 706/54; 726/28

(57) ABSTRACT

A system for monitoring biometric data of an athlete participating in a sporting event comprises at least one biometric sensor carried by the athlete. The sensor senses biometric data for the athlete and delivers the data to a transmitter located on the athlete. The transmitter automatically transmits the biometric data to a wireless telephony network as the athlete participates in the sporting event. The wireless telephony network, in turn, delivers the biometric data to a processing server via the Internet. The processing server receives the biometric data and transforms the data into processed biometric data for the athlete. The processed biometric data for the sporting event is available to the athlete or other authorized individuals in real time and/or any time following the sporting event at a computer connected to the Internet.

### CROSS-REFERENCE TO RELATED APPLICATIONS

This application claims priority from U.S. Provisional Patent Application No. 61/146,559, filed Jan. 22, 2009, the contents of which are incorporated herein by reference.

### FIELD

This application generally relates to physiological data and performance monitoring, and more particularly to systems for sensing, processing and displaying biometric data.

### BACKGROUND

Athletes and their trainers often keep track of the progress and conditioning of the athlete. Many computerized systems exist which collect biometric data from an athlete during training and subsequently process and display such information for use by the athlete or the trainer. Recently, such systems have become available where the sensor designed to collect the biometric data is incorporated into an athletic garment worn by the athlete. An example of such a system is disclosed in U.S. Patent Publication No. 2008/0218310.

With many existing athletic monitoring systems, biometric data for an athlete is collected by a sensing device during a training session. The biometric data collected during the training session is stored in the memory of a computer that is carried by the athlete or within a line of sight of the athlete. For example, an athlete may wear a heart rate monitor during a training session, and data from the heart rate monitor may be transmitted to the memory of a handheld personal computer carried by the athlete (e.g., a wristwatch, portable media player or other handheld personal computer in wired or wireless communication with the sensor). Following the training session, the handheld personal computer is moved to the vicinity of another computer with advanced processing capabilities, such as a desktop or laptop computer. The handheld personal computer is then connected to the additional computer with a wired or wireless connection, and data from the handheld personal computer is downloaded to the additional computer. The additional computer provides the athlete or other user with advanced options not available with the handheld computer alone, including the ability to perform various calculations on the data, view the processed data in various formats, and/or store the data for an extended period of time. Accordingly, with many present systems, data collected during a training session must first be downloaded by manually moving the athlete's handheld personal computer into close proximity with an advanced processing computer before complex calculations and other advanced processing is performed on the data.

One example of an existing monitoring system is represented in FIG. 12. In this system an athlete wears a sensor, such as a heart rate monitor 200, during a training session or other sporting event. The data collected from the heart rate monitor 200 is sent to a transmitter, and the data is then wirelessly transmitted to a watch 202 or other handheld personal computer carried by athlete. In addition, the data may also be transmitted to a team monitoring unit 204, which is attached to a computer having an expanded memory and positioned within a line-of-sight of the athlete or otherwise located in close proximity to the athlete. The team monitoring unit 204 is configured to receive data from multiple sensors worn by different athletes, if applicable, during the sporting event. The athlete may view his or her biometric data activity on the handheld personal computer worn by the athlete. At the completion of the sporting event, the athlete may also choose to manually connect the watch 202 to a personal computer 206, such as a laptop or desktop computer, and download the biometric data received by the watch 202 during the sporting event. The personal computer 206 can then perform further processing on the data for viewing by the athlete. Likewise, a coach, trainer or other individual may manually download the data collected at the team monitoring unit 204 to a remote team computer 208 at the completion of the sporting event. Alternatively, the team computer 208 may be directly connected to the team monitoring unit 204 such that the data is automatically transferred to the team computer 208. The team computer 208 can then perform further processing on the data for viewing by the coach, trainer, athlete, or other individual.

In view of the foregoing, it would be desirable to provide a system and method for sensing biometric data from an athlete during a sporting event and automatically transmitting the biometric data for immediate processing such that the processed data may be viewed in real-time by one or more authorized individuals, on one or more platforms. It would also be advantageous if such a system and method could be utilized in numerous sporting venues. In addition, it would be advantageous if such a system was not limited to line-of-sight data transmissions or data transmissions in close proximity to an athlete being monitored.

### SUMMARY

In accordance with one embodiment of the disclosure, there is provided a system for monitoring biometric data of an athlete participating in a sporting event. In at least one embodiment, the system comprises at least one biometric sensor provided on a garment worn by an athlete. The sensor is configured to collect biometric data for the athlete as the athlete participates in a sporting event. The biometric data collected by the sensor is delivered to a transmitter located on the athlete which automatically transmits the biometric data to a wireless telephony network as the athlete participates in the sporting event. The wireless telephony network, in turn, delivers the biometric data to a processing server via the Internet. The processing server receives the biometric data and transforms it into processed biometric data for the athlete related to the sporting event and the athlete's performance. The processed biometric data for the sporting event is available to the athlete or other authorized individuals in real time and/or any time following the sporting event at a computer connected to the Internet. The computer connected to the Internet may include a desktop computer, laptop computer, handheld computer, cell phone, personal training watch, or any other personal training device worn or carried by the athlete. Because the biometric data collected by the user is automatically transmitted to the Internet via a wireless telephony network, the athlete does not need to manually connect the sensor to a different computer in order to forward the sensed biometric data to the processing computer. Nor is the athlete tied to one single carried device in order to view the data. Instead, the transfer of biometric data occurs automatically during the sporting event without any positive action required by the athlete to facilitate the transfer. This provides the athlete with instantaneous access to the biometric data at any time during or following the sporting event.

Pursuant to another embodiment of the disclosure a method is provided for monitoring at least one athlete participating in a sporting event at a sporting venue. The method includes dressing or otherwise equipping the athlete with a garment having a biometric sensor positioned on the garment. The method further comprises sensing biometric data for at least one athlete as the athlete participates in the sporting event. The sensed biometric data is automatically transmitted during the sporting event to a wireless telephony network via a non line-of-sight transmission from the athlete to a cell tower of the wireless telephony network. The wireless telephony network is connected to the Internet. When the biometric data is received by the wireless telephony network, it is passed on to a processing server via the Internet. The processing server processes the sensed biometric data. The processed biometric data is then delivered back to the sporting venue via the Internet. The processed biometric data for the athlete is viewed by one or more authenticated persons during the sporting event using a computer located at the sporting venue, or remotely from a computer removed from the sporting event. For example, the processed biometric data may be viewed through authentication procedures by coaches, trainers, doctors, scouts, the media or other authorized individuals.

The above described features and advantages, as well as others, will become more readily apparent to those of ordinary skill in the art by reference to the following detailed description and accompanying drawings. While it would be desirable to provide a method and system for monitoring athletic performance that provides one or more of the foregoing or other advantageous features, the teachings disclosed herein extend to those embodiments which fall within the scope of any eventually appended claims, regardless of whether they include one or more of the above-mentioned features or advantages.

### BRIEF DESCRIPTION OF THE DRAWINGS

FIG. 1 shows a front view of an athletic garment and transceiver configured for use in association with a system for monitoring athletic performance;

FIG. 2A shows a diagrammatic view of a system for monitoring athletic performance using the garment of FIG. 1;

FIG. 2B shows a block diagram of another embodiment of the system for monitoring athletic performance of FIG. 2A;

FIG. 3 shows a diagrammatic view of the system of FIG. 2A where a plurality of athletes are monitored with the system;

FIG. 4 shows a diagrammatic view of the system of FIG. 3 where the system is used during a sporting event within a sports venue;

FIG. 5 shows a method of implementing decisions related to a sporting event using the system of FIG. 4;

FIG. 6 shows a first alternative embodiment of the garment and transceiver of FIG. 1;

FIG. 7 shows a second alternative embodiment of the garment and transceiver of FIG. 1;

FIG. 8 shows a third alternative embodiment of the garment and transceiver of FIG. 1;

FIG. 9 shows a fourth alternative embodiment of the garment and transceiver of FIG. 1;

FIG. 10 shows a fifth alternative embodiment of the garment and transceiver of FIG. 1;

FIG. 11 shows a sixth alternative embodiment of the garment and transceiver of FIG. 1; and

FIG. 12 shows a prior art system for monitoring athletic performance.

### DESCRIPTION

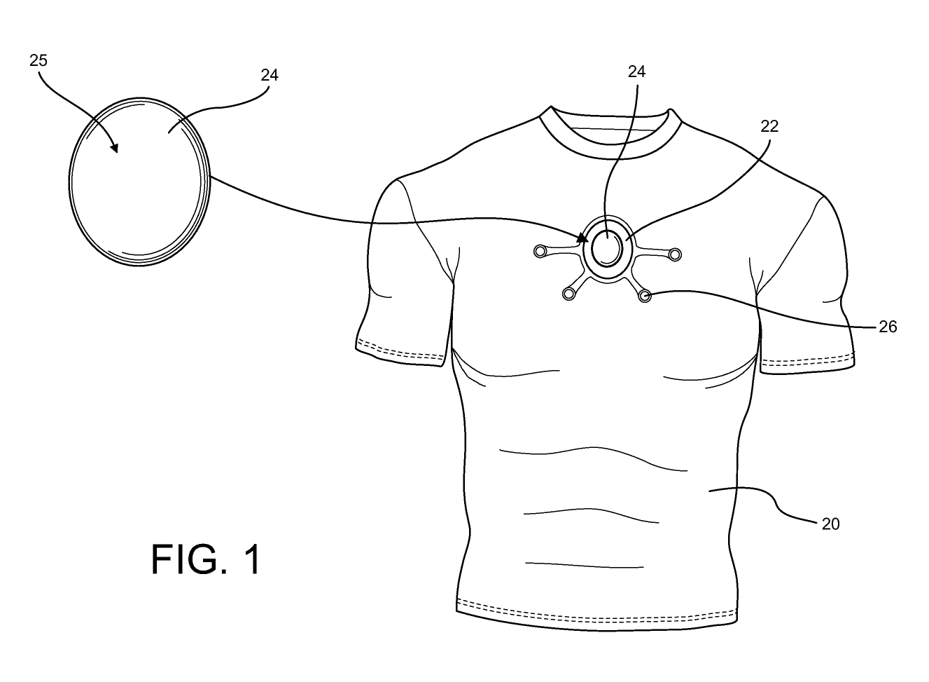

Referring to FIG. 1, there is shown a diagrammatic view of an exemplary embodiment of a system for monitoring an athlete's performance as the athlete participates in a sporting event. The reader should understand that the embodiment discussed herein may be implemented in many alternate forms and variations. Furthermore, the word “sporting event” as used herein refers to any organized or unorganized event where a human participates in a team or individual competition, or a team or individual training session or activity. Examples of “sporting events” include both professional and amateur sports competitions (whether team or individual), team or individual practice sessions to further develop physical skills or prepare for a competition, and/or any team or individual physical workout, physical exercise, athletic conditioning or training session (whether or not in preparation for a competition), or entertainment activity involving physical exertion. The word “sporting venue” as used herein refers to a building, field, street, course, trail, stadium, facility, or any other location where a sporting event occurs. The word “sports stadium” refers specifically to a structure designed to facilitate human viewing of professional or amateur sports competitions with a playing field, floor, course or competition area associated with the sports stadium. The word “athlete” as used herein refers to any human participating in a sporting event. The word “garment” as used herein refers to shirts, shorts, pants, socks, shoes, watches, wristbands, hats, headgear, or any other clothing, footwear, accessory or equipment worn on the human body.

With reference now to FIG. 1, a garment 20 is shown in the form of a shirt. The shirt includes a receptacle 22 configured to hold a communications module 24. At least one sensor 26 is positioned on the shirt or on the athlete wearing the shirt. The sensor is configured to sense biometric data from the athlete wearing the shirt and deliver the sensed biometric data to the transmitter.

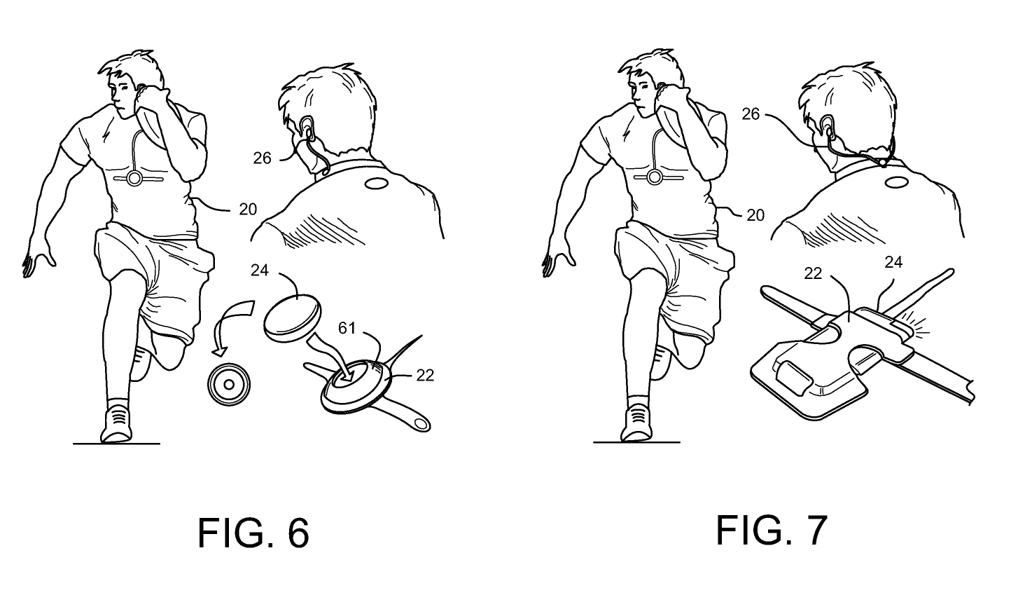

The receptacle 22 on the shirt may be provided in any of numerous forms, such as those described in further detail below with reference to FIGS. 6-11. The receptacle 22 is configured to secure the communications module 24 in place on the garment 20 when it is worn by the user. In at least one embodiment, the receptacle 22 secures the communications module 24 to the garment 20 in a releasable fashion such that the communications module 24 may be removed from the garment by the user without damaging the receptacle or the garment. However, in another alternative embodiment, the communications module 24 may be secured on the garment 20 in a permanent fashion.

The communications module 24 includes electronic circuitry comprising a receiver and a transmitter protected within a durable shell 25 (the electronic circuitry for such transmitters and receivers is known to those of skill in the art and is not shown in the figures). The receiver is configured to receive biometric data signals from the biometric sensors provided on the garment or otherwise carried by the athlete. The transmitter is an rf transmitter configured to transmit received biometric data signals to a wireless telephony network. Accordingly, the communications module also includes a battery configured to power the receiver and the transmitter. In at least one embodiment, the battery of the communications module is a rechargeable battery. In this embodiment, the communications module may be placed in a battery charger configured for use with the communications module in order to recharge the battery. The battery provides the transmitter with sufficient power to transmit an rf signal to a nearby antenna in a wireless telephony network (e.g., about ½ mile to 5 miles or more to an antenna in a mobile telephony network).

The electronics for the communications module 24, including the receiver and transmitter, are housed within the shell 25 to keep the electronics within the communications module safe. Accordingly, the shell 25 may be comprised of a polymer, or fabric material capable of absorbing impacts without damage to the electronics embedded in the shell. Electrical contacts may be provided on the communications module 24 to allow the module 24 to receive biometric data signals delivered from the sensors 26 through a wire. Alternatively the transmitter 24 may be completely enclosed in the shell material and receive the signals from the sensors 26 via a wireless connection. The terms “bug” and “communication bug” are also used herein to refer to the communications module 24. The communications module may be any of various sizes, shapes and configurations, as will be recognized by those of skill in the art.

The sensors 26 include any of numerous biometric sensors that may be used to sense various physiological conditions of the athlete. For example, the biometric sensors 26 may include heart rate sensors, hydration sensors, body temperature sensors, muscle fatigue sensors and numerous other sensors which may be provided in any of various different configurations and arrangements as will be recognized by those of skill in the art. Furthermore, the sensors 26 may also include environmental/positional sensors such as a GPS receiver, air temperature sensor or hygrometer. This data may also be transmitted from the bug to the wireless telephony network. The sensors may be incorporated directly into the garment, housed within the bug 24, or may otherwise be worn or held by the athlete during the sporting event. For example, a heart rate sensor may be embedded in a shirt worn by the athlete or may be worn on a band encircling the athlete's chest. A GPS receiver may be provided directly in the bug, may be fastened to a shirt, or may be provided on a portable media player or telephone clipped to the athlete's waistband. Of course, these are but a few examples of sensors and configurations of sensors that may be used by the athlete in association with the bug. When the sensors are incorporated into the garment 20, they may include electrical connections that lead directly to the receptacle, allowing the bug plugged into the receptacle to receive signals from the sensors 26. Alternatively, the garment 20 may include an electrical connector adapted for connection to other sensors that are not incorporated into the garment. In yet another embodiment, the sensors may each include an associated transmitter that transmits the sensor signal to the bug in a wireless manner.

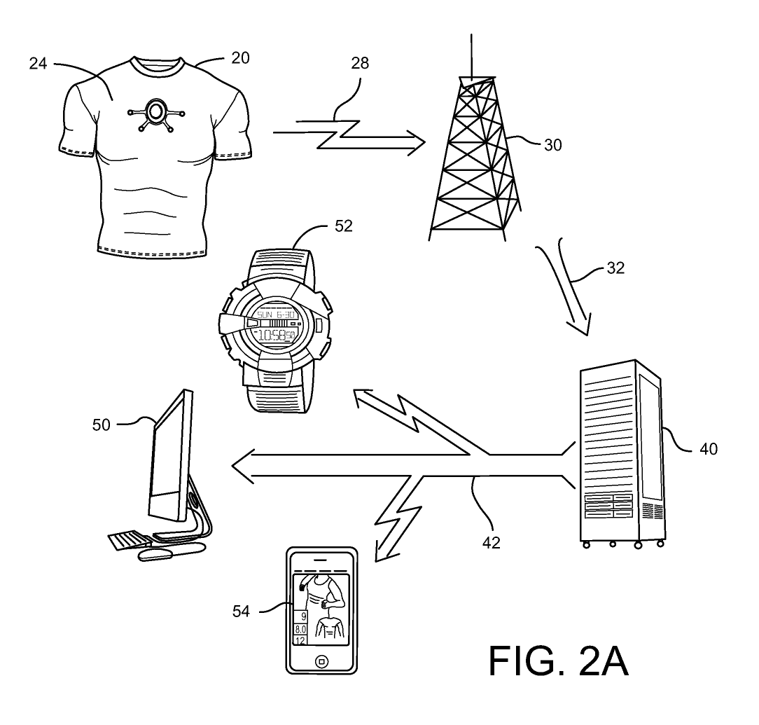

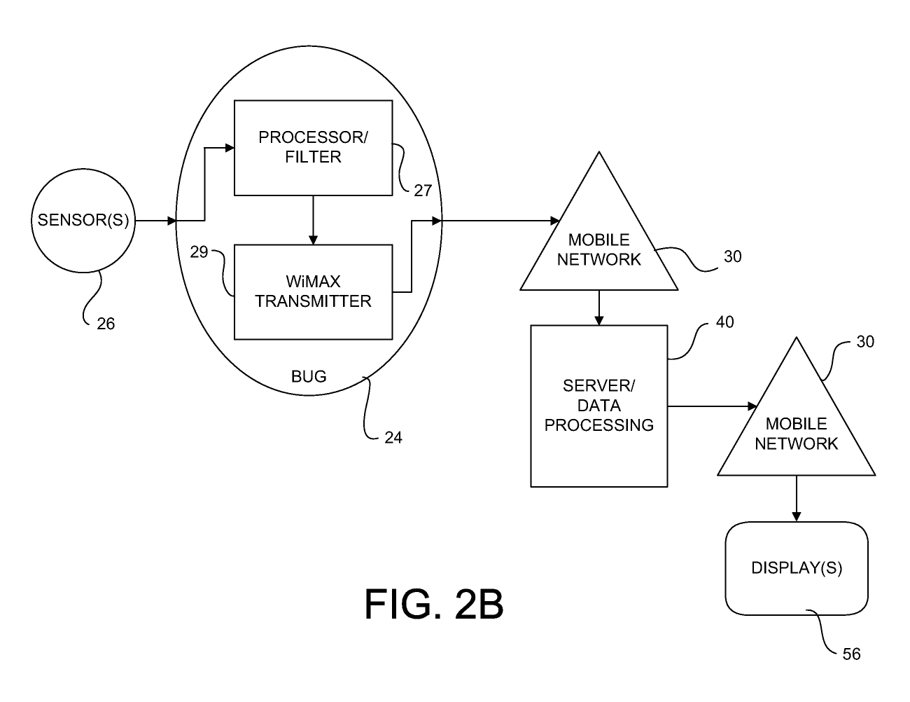

With reference now to FIG. 2A, when an athlete wearing the garment 20 with the attached bug 24 participates in a sporting event, biometric data is delivered to the bug 24 from the sensors 26 worn by the athlete. As represented by arrow 28 the bug is configured to transmit an rf signal representative of the biometric data received by the bug to a wireless telephony network (represented by antenna 30). This transmission from the bug 24 to the wireless telephony network 30 occurs automatically without the athlete needing to prompt the transmission. Because the transmissions are automatic, some mechanism may be used to turn on the bug's transmitter or otherwise indicate that automatic transmissions should begin. For example, in one embodiment, an on/off switch is provided on the bug 24 that allows the athlete to begin automatic transmissions of data from the bug. In another embodiment, the bug 24 may be configured to begin transmissions once it starts receiving biometric data signals from a sensor worn by the athlete. In yet another embodiment, the bug 24 may only begin transmissions once the data signals received from the sensor indicate that an athletic event has started (e.g., increased heart rate or temperature).

In addition to automatic transmissions from the bug, it will also be noted that the transmission of data from the bug to the network 30 occurs in real-time, i.e., at the same time the athlete participates in the sporting event. In one embodiment, the bug 24 transmits biometric data immediately upon receipt of a signal from the sensor worn by the athlete. However, in other embodiments, the bug 24 may be configured to conserve power by only transmitting data in a periodic fashion, such as once every second, once every ten seconds, once every thirty seconds, etc. In these embodiments, the electronics package for the bug 24 may include a memory configured to store a limited amount of data taken over a short period of time and then transmit that data and associated time information in a single transmission. In any event, the system is configured to regularly and automatically transmit data to a wireless telephony network as the athlete participates in the sporting event.

The wireless telephony network 30 shown in FIG. 2A may comprise any of several known or future network types. For example, the wireless telephony network may comprise commonly used cellular phone networks using CDMA or FDMA communications schemes. Some other examples of currently known wireless telephony networks include Wi-Fi, WiMax, GSM networks, as well as various other current or future wireless telecommunications arrangements.

The wireless telephony network 30 is connected to the Internet via the hardware of the particular mobile service provider. As represented by arrow 32, the biometric data received at the antenna 30 of the wireless telephony network is passed on to one or more computers in the form of processing servers 40 via the Internet. In at least one embodiment, each processing server 40 is remotely located from the sporting venue where the athlete is participating in the sporting event. For example, one processing server 40 may be housed at the facilities of the manufacturer of the athletic garment 20, a team, or a related service provider offering data processing services. The processing server 40 may comprise a single Internet server, or a server connected to other computers that perform processing and data storage functions. In at least one alternative embodiment, the processing server 40 may be located at the sporting venue where the athlete is participating in the sporting event. For example, the processing server could be located within the same stadium where an athlete is participating.

With continued reference to FIG. 2A, the processing server 40 collects the raw biometric data received for the athlete wearing the garment 20 and processes the data itself or passes the data to connected computers for processing. The processing computer 40 may perform various calculations on the data and also process the data into any of various forms. Typical calculations performed by the computer might relate to the athlete's current performance, improvement, history, training state, etc. For example, if heart rate data for the athlete is collected, the processing server 40 may plot the data on a graph showing the athlete's heart rate during the entire sporting event. As another example, if body temperature data is collected, the processor may calculate an average body temperature during the sporting event and display the average body temperature on a historical chart of average body temperatures for other sporting events. If GPS data is collected, the speed of the athlete may be calculated over different time periods. Furthermore, the biometric data may be processed into different forms and formats, depending on the particular device that will ultimately be used to view the processed data. For example, the data may be processed into a first format that will allow it to be viewed on a watch and into a second format that will allow it to be viewed on the monitor of a personal computer. While these are but a few examples of how the raw data may be processed, those of skill in the art will recognize that nearly countless other possibilities exist for how the data received from the garment 20 will be processed for subsequent viewing and analysis.

In addition to processing of the raw data itself, the processing server may be further configured to strategically associate additional data with the processed data for presentation to the athlete or other authenticated individual. For example, the processing server may make an analysis of the athlete's progress and recommend a new or different training regime that may assist the athlete in improving in a particular area. Accordingly, the processing server has the ability to act as a virtual personal trainer for the athlete. The fact that the processed data may be delivered to the athlete in real time also allows for suggestions or encouragement to be made to the athlete during the actual sporting event (e.g., an audio clip of “you're doing great” or “your heart rate is too fast—slow down”). Furthermore, because of the server's relatively large processing power, the system provides the ability to continually analyze and learn more about the athlete that is being monitored, such as heart rate patterns during athletic activity. With such learned knowledge, the system is also able to better determine when the athlete's activity is becoming risky or dangerous to his or her health or when the athlete is improving in a particular area.

As indicated by arrow 42, after the raw data is processed by the computer 40, it may be accessed by one of several viewing devices via the Internet. Thus the computer 40 is representative of one or more computers that perform data processing functions and also act as Internet servers.

As shown in FIG. 2A, the processed biometric data available on the server 40 may be accessed and displayed or otherwise presented on a watch 52, portable media player or mobile telephone 54, or any other device that may be worn or held by the athlete and is equipped with an appropriate wireless telephony receiver or other means for connecting to the Internet. Such devices may include screens for viewing the processed biometric data, speakers or other audible output devices for sounding information about the processed biometric data, vibration devices and/or other output devices for transmitting information related to the processed data. In addition, the processed biometric data may be accessed and viewed on a personal computer 50. Each of devices 50, 52 and 54 may be connected to the Internet or wireless telephony network to allow the device to receive the processed data. The personal computer 50 may be connected to the Internet via a wired connection. However, for the portable devices 52, 54 this connection to the Internet may be made using a wireless network (such as a mobile telephony network, Wi-Fi, WiMax, etc.). Of course, the personal computer 50 may also be connected to the Internet via a wireless network. For example if each of the devices 50, 52, and 54 is Wi-Fi enabled, the connection to the Internet may be made so long as the device is in a hotspot. The devices 50, 52 and 54 may also be connected to the Internet using other wireless networks, such as WiMax or traditional cellular telephone networks.

The foregoing arrangement provides for a system where an athlete wearing the garment 20 with bug 24 is connected to the Internet and World Wide Web in real time as he or she actually participates in a sporting event. Because the transmission of the biometric data occurs automatically, there is no need for the athlete to take any particular action to download the data to the Internet during or after participation in an athletic event. For example, there is no need for the user to connect any device to a personal computer at the end of a sporting event for the purpose of downloading biometric data to the processing server. Instead, such transmission of biometric data occurs automatically during the sporting event without the athlete needing to take any positive steps during or after a sporting event in order to transmit the sensed biometric data to a processing server. The transmitted data is automatically processed and available for viewing by the athlete at any time, including during the sporting event, the same day of the sporting event, or several days after the sporting event without any positive steps required by the athlete to download data after the sporting event. Furthermore, because the athlete is connected to the Internet during the actual sporting event and sensed biometric data is automatically transmitted to the processing server, there is no need to process and store as much data on the athlete's body as is stored with current systems. Increased data storage and data processing capabilities are provided at the processing server. Such increased data processing and storage capabilities are simply not possible with prior art systems where data is stored and processed on devices worn by the individual. Furthermore, because the bug 24 on the garment 20 includes a transmitter configured to transmit data using the wireless telephony network 30, the system is configured for use in any location having access to a wireless telephony network. Thus, the system may be used in relatively remote locations. Furthermore, a system is provided where no additional equipment is needed for real time monitoring other than a garment 20 with an associated bug 24 and a viewing device (e.g., 50, 52, 54).

FIG. 2B shows a block diagram of another exemplary embodiment of the system for monitoring athletic performance. The system of FIG. 2B is the same as that of FIG. 2A, but the system is represented in block diagram form and emphasizes that the processed data from the server 40 may also be returned to a display 56 via a wireless telephony network 30. Furthermore, the system of FIG. 2B shows electronic circuitry found within the bug 24. The electronics circuitry includes a processor/filter 27 configured to receive data signals from the sensor(s) 26. The sensor may send data signals to the processor/filter over a wired or wireless connection to the processor filter. The processor/filter includes circuitry that filters noise from the received data signals and then delivers the filtered data signals to a transmitter 29. In the embodiment of FIG. 2B, the transmitter is a WiMax transmitter configured to transmit the filtered data signals to a wireless telephony network 30.

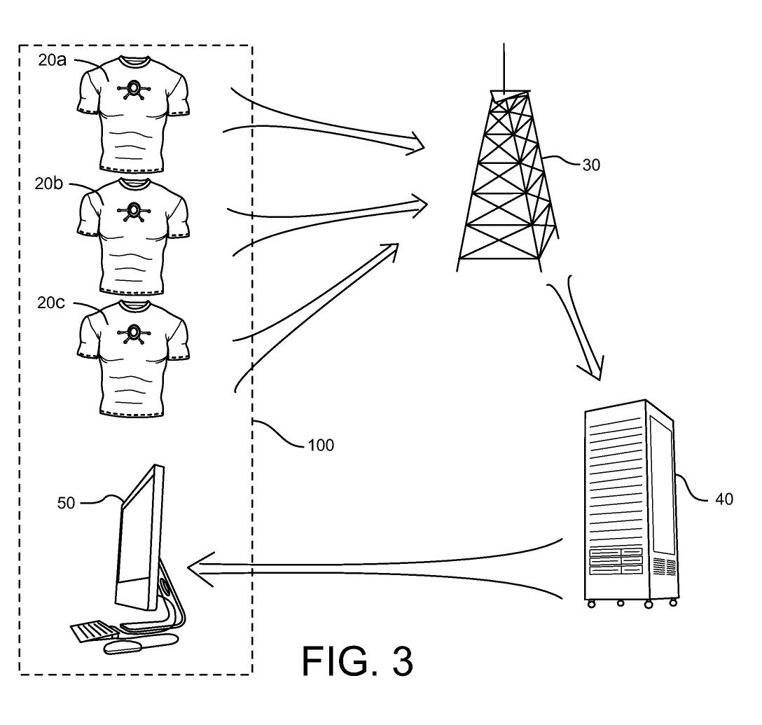

FIG. 3 shows another exemplary embodiment of the system for monitoring athletic performance where the system may be used to monitor a whole team or group of athletes rather than a single athlete. As shown in FIG. 3, multiple garments 20a, 20b and 20c are worn by a team of athletes, each garment 20a, 20b, and 20c including a bug 24. The athletes are all participating in a sporting event at a sporting event venue 100. The bug on each garment 20a, 20b, and 20c transmits data for the athlete to a wireless telephony network 30. That raw data is then passed on to the processing server 40 via the Internet. After the data is processed, it is available for viewing by a coach or a trainer at a computer 50 which is also located at the event venue 100 as shown in FIG. 3. In addition, because the processed data is available via processing server 40, the processed data may also be viewed at remote locations that are not associated with the event venue 100. The processed data may include calculations for the individuals as well as the entire team. Furthermore, the processed data may be used to compare the physiological conditions of a plurality of athletes on the team. Such data may be used by the coach, trainer, or other individual to make decisions related to individuals or the team as a whole. For example, a coach may use the processed biometric data to decide which of two closely matched players should start a game. Numerous other examples are possible as real time and historical performance data for individual athletes and teams is available using the system. This allows data on athletes and teams to be collected over short periods as well as long periods of time, such data collected over many years.

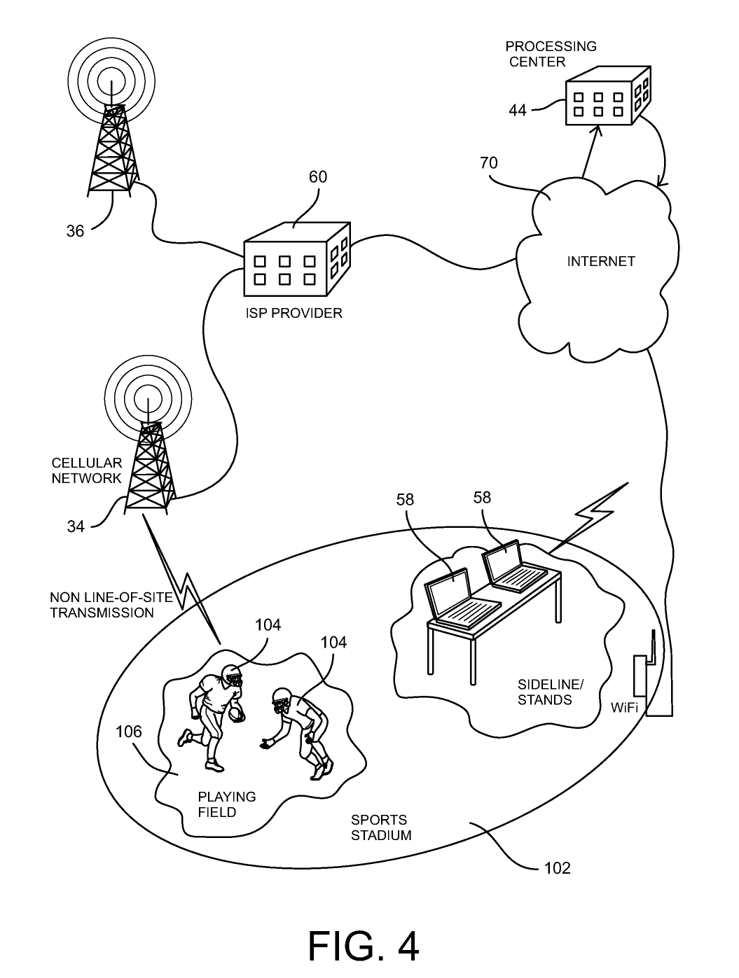

FIG. 4 shows another exemplary embodiment of the system similar to that of FIG. 3 where the system is used in association with a group of athletes. In the particular embodiment of FIG. 4, a professional sports competition is occurring in a sports stadium 102. A plurality of athletes 104 are positioned on a playing field 106. A plurality of laptop computers 58 or handheld personal computers have been brought to the stadium 102 by coaches, trainers, broadcasters and/or fans watching the sporting event. Accordingly, the laptop computers are located throughout the stadium, such as on the sidelines, in the press box, in the stands, in the locker rooms and/or in the training rooms of the stadium. As the athletes participate in the competition, biometric data from the athletes is transmitted to a cellular network 34 including a plurality of antennas 36. The cellular network 30 is connected to the servers of an Internet service provider 60. The Internet service provider 60 is connected to the Internet 70. The servers and other computers of a processing center 44 are also connected to the Internet 70. Accordingly, the biometric data transmitted to the cellular network 34 from the playing field 106 is passed on to the Internet service provider 60 and then to the processing center 44 via the Internet. The biometric data is then processed and used to perform various calculations and prepare reports and other information to be made available for the individuals viewing the competition at the sports stadium 102. Typically, access to such processed data would only be made available to certain authenticated individuals following an authentication procedure, such as entry of a username and password in order to access the processed data. In the embodiment shown in FIG. 4, the processed information may be viewed on the laptop computers 58 located at various locations throughout the stadium 102. In the disclosed embodiment, the laptop computers are WiFi enabled and connected to the Internet via a wireless network. Furthermore, it will also be appreciated that because the processed data may be accessed via the Internet, the processed data may also be made available to other authenticated individuals who are removed from the sports stadium, such as fans watching the competition on television who have paid for a service that allows them to view the authenticated data.

With continued reference to FIG. 4, different processed data may be available to different parties based on authentication/security clearances and procedures such as username and password combinations. For example, the coaches and trainers for one team may have access to all the individual physiological data for the players on that team. Likewise, the coaches and trainers for the opposing team may have access to all the individual physiological data for the players on the opposing team. Sportscasters and others in the broadcast booth or the stands may only have access to more limited information, such as limited physiological data (e.g., heart-rate only) on a single player or a limited number of players.

Using the information made available to those at the sports stadium 102, various decisions affecting physical outcomes may be made. For example, a coach may decide to bench a particular player if the athlete's heart rate is too high, indicating exhaustion and inability to perform. Similarly, the cameras of the broadcaster may focus on a particular player if it is noted that the player has a particular physiological condition, such as an elevated level of perspiration or an elevated heart rate, and the broadcasters may wish to comment on this.

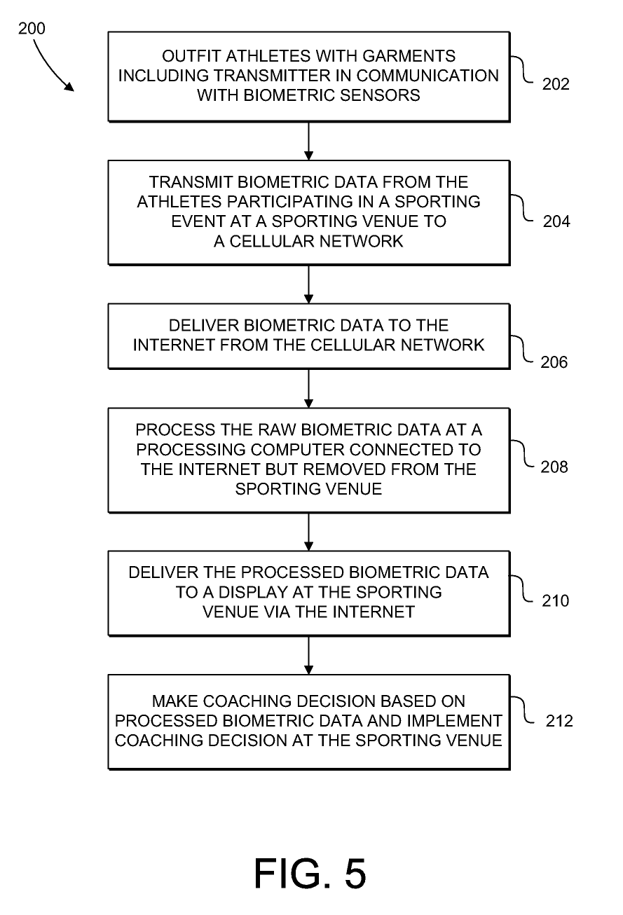

FIG. 5 shows a flowchart related to FIG. 4. The flowchart of FIG. 5 describes a method 200 for monitoring athletic performance using the system shown in FIG. 4. The method 200 begins in step 202 by outfitting athletes with garments 20 carrying a bug 24 and biometric sensors in communication with the bug. In step 204, the bug is used to transmit biometric data from the athletes participating in a sporting event at a sporting venue to a wireless telephony network. In step 206, the biometric data is relayed on to the Internet and delivered to a processing server connected to the Internet. Next, in step 208, the processing server processes the biometric data in order to calculate data about the athlete's performance, history, condition, etc. The processing server processes and stores this information in a location that is removed from the sporting venue. As noted in step 210, the processed data is then delivered to the sports venue via the Internet. Thereafter, in step 212, a coach reviews the processed biometric data and makes a coaching decision based on the data. The coaching step is then implemented during the sporting event at the sporting arena. For example, based on the provided biometric data, the coach may determine that a particular athlete is exhausted and not capable of playing to full potential. The coach may then decide to rest the athlete or temporarily remove the athlete from the sporting event until he or she is rested and ready to return to the game.

It will be recognized that the method of FIG. 5 is but one example of a physical transformation resulting from the system described herein. Another example of such a physical transformation is that of an athlete slowing down during a training exercise in response to a warning from the processing server that the athlete's heart rate is too high. Those of skill in the art will recognize that numerous other physical transformations may also result from use of the system described herein.

FIGS. 6-11 show various embodiments of bugs and receptacles that may be utilized on garments for the above-described system. In FIG. 6 the bug 24 is a disc shaped device retained in a complementary circular polymer receptacle 22 that is fastened to a central location on a shirt 20. The receptacle 22 includes a deformable ring 61 that helps retain the bug 24 in place within the housing, while also allowing the bug 24 to be easily removed from the housing. In the embodiment of FIG. 6, the sensor 26 is an ear temperature sensor with a wired connection to the collar of the shirt 20. An electrical connection extends between the collar and the receptacle allowing sensor data to be relayed from the sensor 26 to the bug 24.

FIG. 7 shows a similar embodiment to that of FIG. 6, but in this embodiment, the receptacle 22 is a pocket configured to receive the bug 26. In particular, the bug 24 is slid downwardly in the pocket until it is properly seated and an electrical connection is established between the bug and the receptacle. Both the pocket and the bug are substantially rectangular in shape.

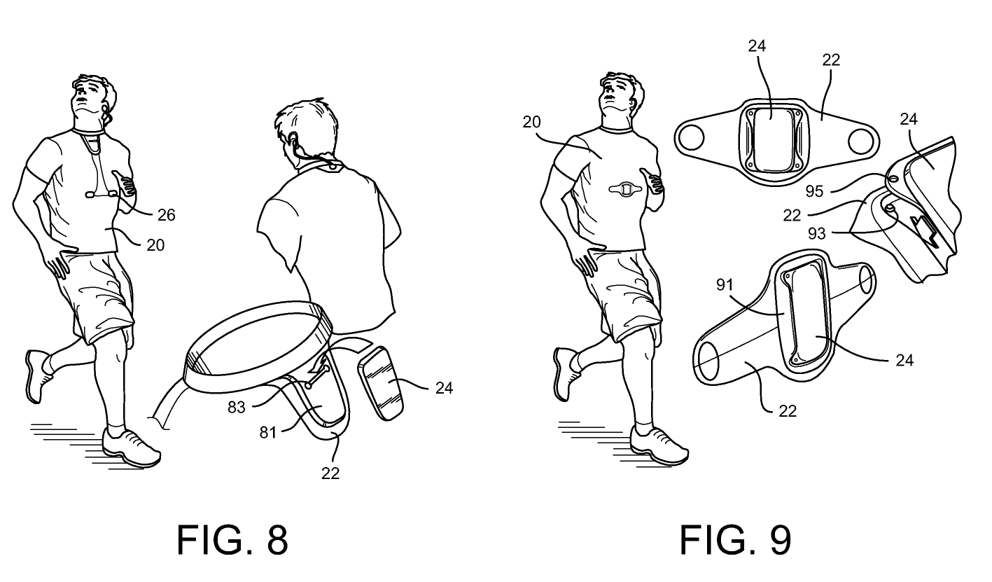

FIG. 8 shows another embodiment of a receptacle 22 and bug 24 combination on a shirt 20. The receptacle 22 is a pocket with a flexible covering 81. A slit opening 83 is provided in the flexible covering 81 which is large enough to receive the bug 24. Electrical connectors are provided on the back side of the bug with complementary connectors provided in the pocket of the receptacle 22. The bug and complementary pocket are both substantially rectangular in shape. The shirt 20 includes sensors 26 (e.g., a heart rate sensor and a hydration sensor) that are fixed upon the shirt and electrically connected to the receptacle 22.

FIG. 9 shows yet another embodiment of a polymer receptacle 22 with an open pocket and flexible side arms 91 that help to retain the bug 24 in place on the receptacle 22. The polymer receptacle may be bonded to the shirt fabric via thermo-plastic adhesive films that are melted using RF welding or heat pressing. Flexible polymer mushrooms 93 may be provided in the receptacle which engage small complementary holes 95 in the bug 24 to secure the bug in place on the receptacle 22. Both the bug and the complimentary pocket are substantially rectangular in shape.

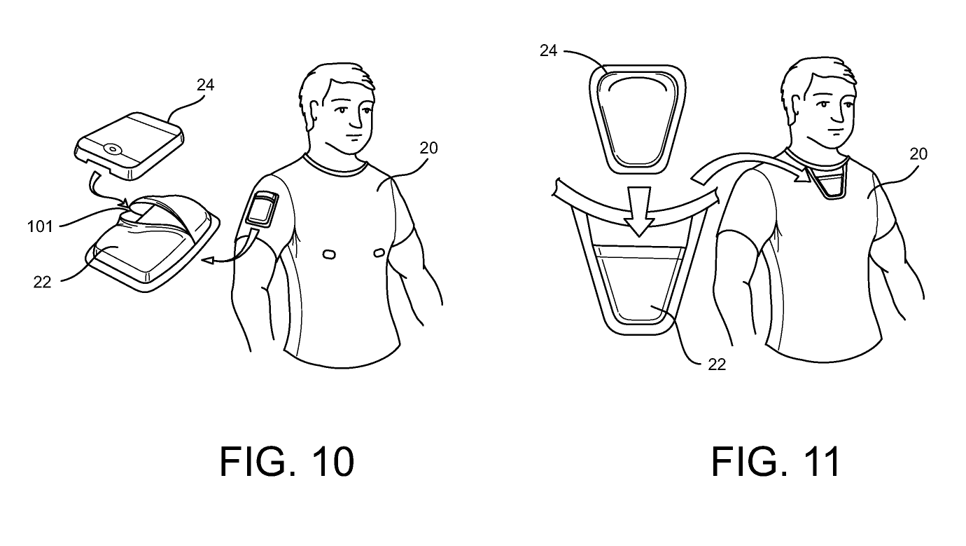

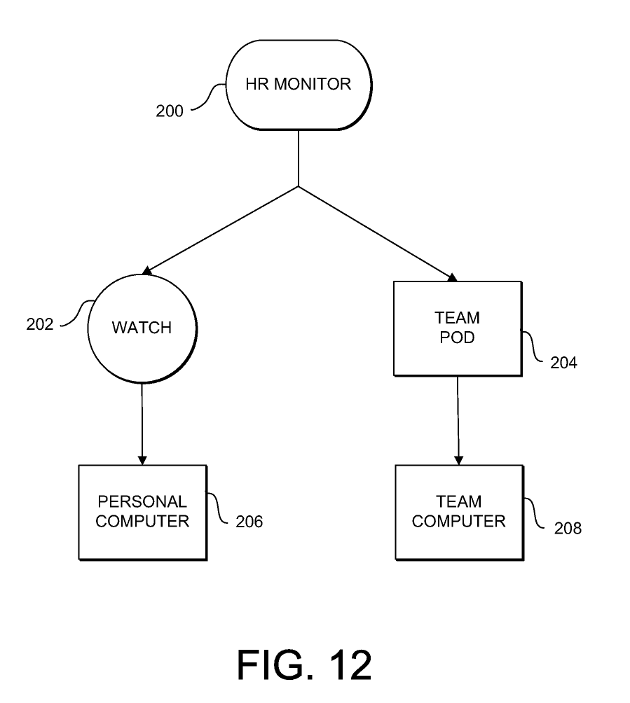

FIG. 10 shows another embodiment of a receptacle 22 and bug 24 combination where the receptacle is a covered polymer, or fabric pocket, similar to that of FIG. 8. However, in FIG. 10, the receptacle is provided on the sleeve of the shirt 20. The receptacle includes an opening 101 formed by two overlapping polymer, or fabric flaps on the pocket. When the flaps are deformed away from each other, the bug 24 may be inserted into the pocket. Once again, both the bug and the complimentary pocket are substantially rectangular in shape.

FIG. 11 shows yet another embodiment of a receptacle 22 and bug 24 combination where the receptacle is only a partially covered polymer, or fabric pocket. In particular, similar to FIG. 7, the bug 24 may be slid into the pocket until it is seated in the pocket and an electrical connection is made between the receptacle and the bug. In the embodiment of FIG. 11, the bug and the pocket are substantially trapezoidal in shape. Furthermore, the pocket 22 is provided on an upper location of the shirt near the collar.

As set forth above, a system and method is provided for monitoring athletic performance. The system provides for the communication of biometric data about an athlete from a communications device carried by the athlete, to the Internet, and optionally, from the Internet to outside devices such as a computer, mobile phone, watch, etc. In at least one embodiment, the device may be removably attached to a compression shirt or other garment that is worn next to the skin. In order to provide this functionality, the communications device gathers data from sensors placed within a garment or shoe, and sends the data via a wireless telephony network such as CDMA, WiMax, GSM, etc., to the Internet, where the data is collected on servers. The data is then processed on the servers to calculate data about an athlete's performance, improvement, history, training state, etc. Using this system, the athlete is automatically linked to the Internet during a sporting event. This removes the need to process and store as much data on the athlete's body, as is done by current systems such as heart-rate/watch combinations. The system allows real-time monitoring by trainers, or scouts remotely, or in-situ; monitoring by the athlete, or monitoring by any party (such as a sportscaster) who has been granted access to the athlete's device.

The ability to automatically send biometric data straight to the Internet during a sporting event provides significant advantages. The disclosed system is flexible and can be used for an individual or by a team with no extra equipment beyond a computer that is equipped with an Internet connection, and garments equipped with the device. Additional functionality can be accomplished by changing the software on the server, rather than upgrading hardware such as a watch. Because the athlete's performance data is stored on a server at all times, the athlete and/or trainers can monitor performance over long periods of time, and over multiple activities. Furthermore, the system provides for automatic cumulative tracking of an athlete during various sporting events and an automated suggestions for improvements (e.g., automated training services available by viewing the processed data).

Although the present invention has been described with respect to certain preferred embodiments, it will be appreciated by those of skill in the art that other implementations and adaptations are possible. Moreover, there are advantages to individual advancements described herein that may be obtained without incorporating other aspects described above. Therefore, the spirit and scope of the appended claims should not be limited to the description of the preferred embodiments contained herein.

What is claimed is:

1. A method of monitoring biometric data comprising:
collecting biometric data using a sensor positioned on an athlete participating in a sporting event;
wirelessly transferring the collected biometric data to a computer in real time using a wireless telephony network; and
processing the biometric data at the computer.

2. The method of claim 1 wherein the sensor is positioned on a garment worn by the athlete during the sporting event.

3. The method of claim 1 wherein the collected biometric data is transmitted to the wireless telephony network using a transmitter worn by the athlete.

4. The method of claim 3 wherein the transmitter is positioned on a garment worn by the athlete.

5. The method of claim 4 wherein the transmission to the wireless telephony network is a non line-of-sight transmission from the athlete to a cell tower of the wireless telephony network.

6. The method of claim 1 wherein the wireless telephony network is connected to the Internet and the collected biometric data is transferred from the wireless telephony network to the computer via the Internet.

7. The method of claim 1 further comprising transmitting the processed biometric data to a sporting venue where the athlete is located during the sporting event.

8. The method of claim 7 further comprising viewing the processed biometric data on a screen at the sporting venue during the sporting event.

9. The method of claim 8 further comprising making a decision or recommendation related to the participation of the athlete in the sporting event based on the processed biometric data.

10. The method of claim 7 further comprising automatically informing the athlete of his or her performance level during the sporting event or recommending an action for the athlete during the sporting event using an audible output device worn by the athlete.

11. A system for monitoring biometric data of an athlete participating in a sporting event, the system comprising:
at least one biometric sensor mounted on a garment worn by the athlete, the sensor configured to collect biometric data for the athlete as the athlete participates in a sporting event;
a transmitter provided on the garment, wherein the biometric data collected by the sensor is automatically transmitted by the transmitter to a wireless telephony network as the athlete participates in the sporting event;
a processing server configured to receive the biometric data from the wireless telephony network via the Internet, the processing server configured to transform the received biometric data into processed biometric data for the athlete, the processed biometric data related to the athlete's performance in the sporting event, wherein the processed biometric data for the athlete is available in real time and/or a time following the sporting event at a computer connected to the Internet.

12. The system of claim 11 wherein the computer connected to the Internet comprises a desktop computer, a laptop computer, a handheld computer, or a watch positioned at a sporting venue where the athlete is participating in the sporting event.

13. The system of claim 11 wherein the computer connected to the Internet comprises a computer carried by the athlete during the sporting event.

14. The system of claim 11 wherein the garment is a shirt and the at least one biometric sensor is positioned in a flexible pocket on the shirt.

15. A method for monitoring at least one athlete participating in a sporting event at a sporting venue, the method comprising:
sensing biometric data for at least one athlete as the athlete participates in the sporting event at the sporting venue;
automatically transmitting the sensed biometric data during the sporting event to a wireless telephony network via a non line-of-sight transmission from the athlete to a cell tower of the wireless telephony network positioned outside of the sporting venue, wherein the wireless telephony network is connected to the Internet;
receiving the sensed biometric data at a server connected to the wireless telephony network via the Internet;
processing the sensed biometric data at the server or a computer connected to the server; and
displaying the processed biometric data on a portable computer located at the sporting venue.

16. The method of claim 15 further comprising transmitting the processed biometric data from the server to the portable computer located at the sporting venue via the Internet.

17. The method of claim 15 further comprising performing an authentication procedure to confirm that at least one person viewing the displayed processed biometric data is an authenticated person.

18. The method of claim 15 wherein the athlete is a member of a team and the authenticated person is a coach, trainer or doctor associated with the team or is a member of the media.

19. The method of claim 15 wherein the biometric data is sensed using a biometric sensor positioned on a garment worn by the athlete.

20. The method of claim 15 wherein the server or the computer connected to the server is the same as the portable computer located at the sporting venue.
